(19) United States
(12) Patent Application Publication
(10) Pub. No.: US 2019/0233876 A1
(43) Pub. Date: Aug. 1, 2019

(54) ANTIGEN-COUPLED HYBRIDIZATION REAGENTS

(71) Applicant: CELL IDX, INC., San Diego, CA (US)

(72) Inventor: David A. SCHWARTZ, Encinitas, CA (US)

(73) Assignee: CELL IDX, INC., San Diego, CA (US)

(21) Appl. No.: 16/318,801

(22) PCT Filed: Jul. 18, 2017
(86) PCT No.: PCT/US2017/042659

§ 371 Date: Jan. 18, 2019

Related U.S. Application Data
(60) Provisional application No. 62/363,825, filed on Jul. 18, 2016.

(51) Int. Cl. C12Q 1/6804; C12Q 1/6841; C12Q 1/6876; C12Q 1/6816; C07K 16/18; C12Q 1/28; C12Q 1/42

(52) U.S. Cl. CPC C12Q 1/6804; C12Q 1/6841; C12Q 1/6876; C12Q 1/6816; C07K 16/18; C12Q 1/28; C12Y 301/03004; C12Q 2543/10; C12Q 2563/131; C07K 2317/92; C12Y 101/03004; C12Y 111/01; C12Q 1/42

(57) ABSTRACT

The present disclosure provides high-performance hybridization reagents for use in a variety of hybridization assays and other related techniques. The hybridization reagents comprise an oligonucleotide probe and a bridging antigen, wherein the bridging antigen is recognized by a detectable antibody with high affinity. Also provided are compositions comprising panels of hybridization reagents specific for multiple different target nucleic acids and compositions comprising pairs of hybridization reagents and their complementary detectable antibodies. The paired hybridization reagents and detectable antibodies are useful in a variety of hybridization assays, particularly in highly multiplexed assays, where the structure of the bridging antigen is varied in tandem with variation in the detectable antibody, such that a multiplicity of hybridization reagents are provided that are capable of simultaneously detecting a multiplicity of target nucleic acids in a single assay. Also provided are kits comprising the hybridization reagents, methods of hybridization assay using the hybridization reagents of the disclosure, and methods of preparation of the hybridization reagents.

### CROSS-REFERENCE TO RELATED APPLICATIONS

This application claims the benefit of U.S. Provisional Application No. 62/363,825, filed on Jul. 18, 2016, the disclosure of which is incorporated herein by reference in its entirety.

### BACKGROUND OF THE INVENTION

The ability to detect low-expressing target markers, including proteins and nucleic acids, in some cases at less than picogram levels, in cellular assays with high sensitivity and specificity continues to be an unmet need. Such approaches become even more important as the sample size of cells and tissues available for analysis becomes smaller and smaller. Furthermore, the ability to simultaneously detect multiple low-expressing targets in a single assay would be of further benefit.

In situ hybridization (ISH) is a powerful labeling technique used to detect nucleic acids in a biological sample. The general method typically involves the use of a labeled DNA, RNA, or modified nucleic acid probe that is complementary to the target nucleic acid of interest in a fixed tissue or cellular sample. The labeled probe is hybridized to the target DNA or RNA sequence in the sample, thus providing temporal and spatial information about one or more genetic loci (e.g., for genomic DNA targets) or one or more expressed genes (e.g., for RNA targets).

In situ hybridization techniques can be distinguished from one another according to the type of label used to modify the probe and consequently the detection method used to identify the target. For chromogenic in situ hybridization (CISH) assays, peroxidase or alkaline phosphatase reactions, such as the reactions and labels conventionally used in IHC staining, are used to generate a chromogenic signal at the location of the target. The signal is subsequently visualized using bright-field microscopy. CISH can be used to measure, for example, gene amplification, gene deletion, chromosome translocation, and chromosome number. It can be applied in particular, to formalin-fixed, paraffin-embedded (FFPE) tissues, metaphase chromosome spreads, fixed cells, and blood or bone marrow smears.

For fluorescent in situ hybridization (FISH) assays, fluorescent labels are used in the detection process, and the signals are detected using fluorescence microscopy or related spectroscopic techniques. The use of multiple fluorescent labels, with spectrally distinct fluorescence properties, enables the simultaneous detection and colocalization of multiple nucleic acid targets within a single sample. For DNA targets, FISH can therefore be used, for example, to detect the presence, copy number, and location of genomic loci of interest and to identify gene mutants and chromosomal defects. For RNA targets, FISH can be used, for example, to assess gene expression, both temporally and spatially, thus providing insights into physiological processes and disease pathogenesis.

In situ hybridization techniques can additionally be used in combination with immunohistochemical (IHC) staining techniques to label simultaneously target nucleic acids and expressed target proteins in a tissue sample or on another suitable surface.

Despite the usefulness of the above approaches, however, there continues to be a need for the development of improved hybridization assay reagents, methods, and kits that are more sensitive, more specific, and more able to detect multiple nucleic acid targets in a single assay.

### SUMMARY OF THE INVENTION

The present disclosure addresses these and other needs by providing in one aspect a hybridization reagent composition that finds utility in a variety of hybridization assays. Specifically, according to this aspect of the invention, the hybridization reagent composition comprises:
an oligonucleotide probe coupled to a bridging antigen; and a detectable antibody;
   wherein the detectable antibody is specific for the bridging antigen with high affinity.

In some embodiments, the bridging antigen is a peptide or small-molecule hapten.

In some embodiments, the bridging antigen comprises a plurality of antigenic determinants. In specific embodiments, each antigenic determinant in the plurality of antigenic determinants is the same. In other specific embodiments, the plurality of antigenic determinants comprises a linear repeating structure. More specifically, the linear repeating structure is a linear repeating peptide structure.

In other specific embodiments, the plurality of antigenic determinants comprises at least three antigenic determinants or the bridging antigen comprises a branched structure.

In some embodiments, the bridging antigen is a peptide comprising a non-natural residue. Specifically the non-natural residue may be a non-natural stereoisomer or a β-amino acid.

In some embodiments, the oligonucleotide probe and the bridging antigen are coupled by a chemical coupling reaction through a conjugation moiety. In specific embodiments, the oligonucleotide probe and the bridging antigen are coupled by a high-efficiency conjugation moiety. In some of these embodiments, the high-efficiency conjugation moiety is a Schiff base, such as a hydrazone or an oxime. In some embodiments, the high-efficiency conjugation moiety is formed by a click reaction. In some embodiments, the conjugation moiety comprises a cleavable linker.

In embodiments, the detectable antibody comprises a detectable label. In some embodiments, the detectable label is a fluorophore, an enzyme, an upconverting nanoparticle, a quantum dot, or a detectable hapten. In specific embodiments, the detectable label is a fluorophore. In other specific embodiments, the enzyme is a peroxidase, such as a horseradish peroxidase or a soybean peroxidase, is an alkaline phosphatase, or is a glucose oxidase.

According to some embodiments, the detectable antibody is specific for the bridging antigen with a dissociation constant of at most 100 nM, at most 30 nM, at most 10 nM, at most 3 nM, at most 1 nM, at most 0.3 nM, at most 0.1 nM, at most 0.03 nM, at most 0.01 nM, at most 0.003 nM, or even lower.

Some composition embodiments comprise a plurality of bridging antigen-coupled oligonucleotide probes and a plurality of detectable antibodies, including compositions comprising three, five, ten, or even more reagent pairs.

In another aspect, the disclosure provides immunoreagents comprising:

an oligonucleotide probe coupled to a bridging antigen.

In specific embodiments, the hybridization reagents include one or more of the features of the hybridization reagents of the above-described hybridization reagent compositions.

According to another aspect, the disclosure provides multiplexed hybridization reagent compositions comprising a plurality of any of the above-described hybridization reagents. In specific embodiments, the compositions comprise at least three, at least five, at least ten, or even more of the hybridization reagents.

In another aspect, the disclosure provides methods for hybridization assay comprising:

providing a first sample comprising a first target nucleic acid;

reacting the first target nucleic acid with a first hybridization reagent, wherein the first hybridization reagent is any of the above hybridization reagents complementary to the first target nucleic acid;

reacting the first hybridization reagent with a first detectable antibody, wherein the first detectable antibody is specific for the bridging antigen of the first hybridization reagent with high affinity; and

detecting the first detectable antibody that is associated with the bridging antigen of the first hybridization reagent.

In specific embodiments, the first detectable antibody comprises a detectable label. More specifically, the detectable label may be a fluorophore, an enzyme, an upconverting nanoparticle, a quantum dot, or a detectable hapten. In some embodiments, the detectable label is a fluorophore, and in some embodiments, the enzyme is a peroxidase, an alkaline phosphatase, or a glucose oxidase. In specific embodiments, the peroxidase is a horseradish peroxidase or a soybean peroxidase.

In some embodiments, the first target nucleic acid is within a tissue section. In these embodiments, the detecting step may be a fluorescence detection step or an enzymatic detection step.

In some embodiments, the first target nucleic acid may be in or on a cell. In these embodiments, the first target nucleic acid may be in the cytoplasm of the cell or in the nucleus of the cell.

In some embodiments, the detecting step is a fluorescence detection step, and in specific embodiments, the method may further comprise the step of sorting cells that have bound the first detectable antibody.

In some embodiments, the methods further comprise

reacting a second target nucleic acid on the first sample with a second hybridization reagent, wherein the second hybridization reagent is any of the above hybridization reagents complementary to the second target nucleic acid;

reacting the second hybridization reagent with a second detectable antibody, wherein the second detectable antibody is specific for the bridging antigen of the second hybridization reagent with high affinity; and

detecting the second detectable antibody that is associated with the bridging antigen of the second hybridization reagent.

More specific method embodiments further comprise detecting at least three target nucleic acids in the sample, at least five target nucleic acids in the sample, or even at least ten target nucleic acids in the sample.

Some method embodiments further comprise the steps of:

reacting a second target nucleic acid on a second sample with a second hybridization reagent, wherein the second hybridization reagent is any of the above hybridization reagents complementary to the second target nucleic acid;

reacting the second hybridization reagent with a second detectable antibody, wherein the second detectable antibody is specific for the bridging antigen of the second hybridization reagent with high affinity; and

detecting the second detectable antibody that is associated with the bridging antigen of the second hybridization reagent; wherein the first sample and the second sample are serial sections of a tissue sample.

Other method embodiments comprise the steps of:

providing a sample comprising a first target nucleic acid;

reacting the first target nucleic acid with a first hybridization reagent, wherein the first hybridization reagent is any of the above hybridization reagents complementary to the first target nucleic acid;

reacting the first hybridization reagent with a first reactive antibody, wherein the first reactive antibody binds to the bridging antigen of the first hybridization reagent with high affinity; and

reacting the first reactive antibody with a first detectable reagent, wherein the first detectable reagent is bound to the sample in proximity to the first target nucleic acid.

In some embodiments, these methods further comprise the step of:

dissociating the first reactive antibody from the sample.

In some embodiments, these methods still further comprise the steps of:

reacting a second target nucleic acid on the sample with a second hybridization reagent, wherein the second hybridization reagent is any of the of the above hybridization reagents complementary to the second target nucleic acid;

reacting the second hybridization reagent with a second reactive antibody, wherein the second reactive antibody binds to the bridging antigen of the second hybridization reagent with high affinity; and

reacting the second reactive antibody with a second detectable reagent, wherein the second detectable reagent is bound to the sample in proximity to the second target nucleic acid.

In some embodiments, these methods comprised the step of:

detecting the first detectable reagent and the second detectable reagent on the sample.

According to another aspect, the disclosure provides kits for hybridization assay. In embodiments, the kits comprise any of the above hybridization reagents, a detectable antibody specific for the bridging antigen of the hybridization reagent with high affinity, and instructions for using the kit. In specific embodiments, the kits comprise at least three, at least five, or even at least ten of any of the above hybridization reagents; at least three, at least five, or even at least ten detectable antibodies specific for the bridging antigens of the hybridization reagents with high affinity; and instructions for using the kit.

### DETAILED DESCRIPTION OF THE INVENTION

### Antigen-Coupled Hybridization Reagents

The instant disclosure provides in one aspect high-performance hybridization reagents comprising an oligonucleotide probe and a bridging antigen, wherein the oligonucleotide probe and the bridging antigen are coupled, and wherein the bridging antigen is recognizable by a high-affinity detectable antibody.

The instant hybridization reagents may be used in hybridization assays to identify and bind to a target nucleic acid of interest in the assay, where the specificity of target binding is determined by the complementarity of the oligonucleotide probe used to prepare the hybridization reagent. In particular, the oligonucleotide probes of the instant hybridization reagents may be directed to a target nucleic acid of interest, including deoxyribonucleic acid (DNA), ribonucleic acid (RNA), and any of their natural or synthetic variants, without limitation, either within a cell or in a cell-free system. The target nucleic acid may in some cases be found within a subcellular organelle, for example within the nucleus of a cell or within the mitochondria. The target nucleic acid may alternatively be displayed on a surface of interest, such as, for example, on a nucleic acid blot or other type of two-dimensional medium. The target nucleic acid may in some cases be in impure form, in partly purified form, or in purified form. In general, the target nucleic acid may be on or in any suitable surface, or may even be free in solution, so long as it is available to interact specifically with the hybridization reagent.

The bridging antigen of the instant hybridization reagents is chosen to be recognizable by a secondary antibody, ideally at high affinity. The structure of the bridging antigen is therefore limited only by molecules that are capable of eliciting an immune response in a suitable animal or that can be used to generate suitable secondary antibodies by another means.

In some embodiments, the bridging antigen and the oligonucleotide probe are prepared separately and are attached to one another by a chemical coupling reaction. In these embodiments, the bridging antigen is designed to contain at least one group capable of chemically coupling the bridging antigen to the oligonucleotide probe of the hybridization reagent. As described in more detail below, the coupling group may be chosen, in specific embodiments, so that the bridging antigen is conjugated to the oligonucleotide probe with high specificity and efficiency. In addition, coupling of the bridging antigen to the oligonucleotide probe should not significantly affect the ability of the bridging antigen to be recognized by the detectable antibody. It is also desirable that the bridging antigen and coupling group not themselves have interfering absorbance or fluorescence, so as to avoid any background signals. Furthermore, bridging antigens and coupling groups should be available at high purity and ideally at low cost.

In some embodiments, the bridging antigen of the instant disclosure is a synthetic bridging antigen. In some embodiments, the bridging antigen is a natural product. In specific embodiments, the bridging antigen is a peptide.

Peptides, either synthetic or isolated from natural sources, have been used extensively to generate specific, high-affinity antibodies by various means, as is widely known and understood by those of ordinary skill in the art. The range of structural variation possible with peptides is nearly limitless, thus making them ideally suited for use as bridging antigens in the instant hybridization reagents. Furthermore, synthetic peptides can be designed to include reactive groups to facilitate their coupling to oligonucleotide probes, for example by including amino acid residues or other linking moieties incorporated on the C- or N-termini or internally during solid phase peptide synthesis or post-synthetically with desirable reactive properties within the peptide sequence. Peptidic bridging antigens may be of any size and may contain any suitable amino acids or other residue, both natural and artificial. They may be linear or circular. The peptidic bridging antigens are limited in these embodiments only by their ability to be conjugated to an antibody of interest and to be recognizable by a detectable antibody.

In some embodiments, the bridging antigen is a peptide comprising a non-natural residue. For example, the bridging antigen may comprise a non-natural stereoisomer, such as a D-amino acid. In some embodiments, the non-natural residue may be a non-natural amino acid, such as a β-amino acid or the like. In some embodiments, the residues of the bridging antigen may be coupled using non-peptidic bonding, as would be understood by those of ordinary skill in the art.

Other suitable bridging antigens usefully included in the instant hybridization reagents include non-peptidic small-molecule antigens. As was true with peptidic bridging antigens, such antigens are limited only by their ability to be coupled to an oligonucleotide probe and to be recognizable by a detectable antibody. Exemplary non-peptidic, small-molecule antigens, which may also be referred to herein as “haptens”, include without limitation molecules such as nitrophenyl, dinitrophenyl, trinitrophenyl, digoxygenin, biotin, 5-bromodeoxyuridine, 3-nitrotyrosine, small-molecule drugs, and any other similar chemical tag.

In order to increase the number of binding sites per hybridization reagent, it may be advantageous in some cases for a single bridging antigen to comprise a plurality of antigenic determinants or epitopes. Multiplicity of antigenic determinants in a bridging antigen may increase the number of secondary antibodies able to bind to the hybridization reagent and thus the sensitivity of assays using the hybridization reagent. In some embodiments, the plurality of antigenic determinants may comprise multiple copies of the same antigenic determinant, whereas in some embodiments, the plurality of antigenic determinants may comprise different antigenic determinants. In some embodiments, the plurality of antigenic determinants may comprise a linear repeating structure. More specifically, the linear repeating structure may be a linear repeating peptide structure. In some embodiments, the plurality of antigenic determinants may comprise at least two antigenic determinants, at least three antigenic determinants, at least four antigenic determinants, at least six antigenic determinants, or even more antigenic determinants.

In some embodiments, the bridging antigen may comprise a branched structure. For example, the branched structure may comprise a dendrimeric structure or the like, such as, for example, other polymerized constructs, as would be understood by those of ordinary skill in the art.

Furthermore, it should be understood that a bridging antigen comprising a plurality of antigenic determinants may comprise one or more polyethylene glycol linkers, and the like, between the antigenic determinants, for example between peptide antigenic determinants.

In some embodiments, the peptide antigenic determinants comprise at least four, at least six, at least eight, at least ten, at least 15, at least 20, or even more amino acid residues per antigenic determinant.

Where the oligonucleotide probe and bridging antigen are prepared from separate molecular entities, it should be understood that the coupling of the oligonucleotide probe and the bridging antigen may be achieved in a wide variety of ways, depending on the desired outcome. If control of the location and degree of coupling of the bridging antigen to the oligonucleotide probe is not important, non-specific chemical cross-linkers may be used to achieve the coupling. It is generally desirable, however, for the bridging antigen to be coupled to the oligonucleotide probe in a controlled and specific manner, and the choice of coupling method and agent can affect the location, degree, and efficiency of the coupling. For example, although reactive thiol and amino groups are not found naturally in nucleic acids, they can be included at various locations within an oligonucleotide probe during the synthesis of the oligonucleotide in order to provide a specific location for the attachment of a thiol- or amino-reactive bridging antigen.

In some hybridization reagent embodiments, the oligonucleotide probe and the bridging antigen are coupled by a chemical coupling reaction through a conjugation moiety. In specific embodiments, the oligonucleotide probe and the bridging antigen are coupled by a high-efficiency conjugation moiety. Because the hybridization reagents are preferably synthesized with relatively low molar concentrations of starting materials, and because those starting materials may be expensive and available in relatively small chemical quantities, it is highly desirable that formation of the conjugation moiety be as efficient and specific as possible and that its formation is complete, or nearly complete, at low molar concentrations of reactants. Specifically, it is desirable that the conjugation moiety be capable of coupling an oligonucleotide probe and a bridging antigen with rapid kinetics and/or high association constants and that the association reaction therefore be as efficient as possible in terms of its completion.

The high-efficiency conjugation moieties of the instant hybridization reagents are typically formed, as described in more detail below, by separate modification of each component of the hybridization reagent with complementary conjugating reagents. The complementary conjugating reagents additionally include a further reactive moiety, for example a thiol-reactive or an amino-reactive moiety, that allows the conjugating reagents to be attached to the relevant hybridization reagent component, for example to the oligonucleotide probe and to the bridging antigen. After the oligonucleotide probe and the bridging antigen have been modified by the respective complementary conjugating reagents, the complementary conjugating features on the modified components associate with one another in a highly efficient and specific manner to form the conjugation moiety.

Depending on the situation, the high-efficiency conjugation moiety of the instant hybridization reagents may be a covalent or non-covalent conjugation moiety. In specific embodiments, the high-efficiency conjugation moiety is a covalent conjugation moiety, for example, a hydrazone, an oxime, or another suitable Schiff base moiety. Non-limiting examples of such conjugation moieties may be found, for example, in U.S. Pat. No. 7,102,024, which is incorporated by reference herein in its entirety for all purposes. These conjugation moieties may be formed by reaction of a primary amino group on the conjugating reagent attached to one component of the hybridization reagent (e.g., a synthetic oligonucleotide probe modified with an amino group) with a complementary carbonyl group on the conjugating reagent attached to the other component of the immunoreagent (e.g., a bridging antigen).

For example, hydrazone conjugation moieties may be formed by the reaction of a hydrazino group, or a protected hydrazino group, with a carbonyl moiety. Exemplary hydrazino groups include aliphatic, aromatic, or heteroaromatic hydrazine, semicarbazide, carbazide, hydrazide, thiosemicarbazide, thiocarbazide, carbonic acid dihydrazine, or hydrazine carboxylate groups. See U.S. Pat. No. 7,102,024. Oxime conjugation moieties may be formed by the reaction of an oxyamino group, or a protected oxyamino group, with a carbonyl moiety. Exemplary oxyamino groups are described below. The hydrazino and oxyamino groups may be protected by formation of a salt of the hydrazino or oxyamino group, including but not limited to, mineral acid salts, such as but not limited to hydrochlorides and sulfates, and salts of organic acids, such as but not limited to acetates, lactates, malates, tartrates, citrates, ascorbates, succinates, butyrates, valerates and fumarates, or any amino or hydrazino protecting group known to those of skill in the art (see, e.g., Greene et al. (1999) Protective Groups in Organic Synthesis (3rd Ed.) (J. Wiley Sons, Inc.)). The carbonyl moiety used to generate a Schiff base conjugation moiety is any carbonyl-containing group capable of forming a hydrazone or oxime linkage with one or more of the above hydrazino or oxyamino moieties. Preferred carbonyl moieties include aldehydes and ketones, in particular aromatic aldehydes and ketones. In preferred embodiments of the instant disclosure, the high-efficiency conjugation moiety is formed by the reaction of an oxyamino-containing component and an aromatic aldehyde-containing component in the presence of aniline catalysis (Dirksen et al. (2006) Angew. Chem. 45:7581-7584 (DOI: 10.1002/anie.200602877).

The high-efficiency conjugation moiety of the instant immunoreagents may alternatively be formed by a “click” reaction, for example the copper-catalyzed reaction of an azide-substituted component with an alkyne-substituted component to form a triazole conjugation moiety. See Kolb et al. (2001) Angew. Chem. Int. Ed. Engl. 40:2004; Evans (2007) Aus. J. Chem. 60:384. Copper-free variants of this reaction, for example the strain-promoted azide-alkyne click reaction, may also be used to form the high-efficiency conjugation moiety. See, e.g., Baskin et al. (2007) Proc. Natl Acad. Sci. U.S.A. 104:16793-97. Other click reaction variants include the reaction of a tetrazine-substituted component with either an isonitrile-substituted component (Stockmann et al. (2011) Org. Biomol. Chem. 9:7303) or a strained alkene-substituted component (Karver et al. (2011) Bioconjugate Chem. 22:2263).

The basic features of a click reaction are well understood by those of ordinary skill in the art. See Kolb et al. (2001) Angew. Chem. Int. Ed. Engl. 40:2004. Useful click reactions include generally but are not limited to [3+2] cycloadditions, such as the Huisgen 1,3-dipolar cycloaddition, and in particular the Cu(I)-catalyzed stepwise variant, thiol-ene click reactions, Diels-Alder reactions and inverse electron demand Diels-Alder reactions, [4+1] cycloadditions between isonitriles (isocyanides) and tetrazines, nucleophilic substitutions, especially to small strained rings like epoxy and aziridine compounds, carbonyl-chemistry-like formation of ureas, and some addition reactions to carbon-carbon double bonds. Any of the above reactions may be used without limitation to generate a covalent high-efficiency conjugation moiety in the instant hybridization reagents.

In some embodiments, the conjugation moiety of the instant hybridization reagents comprises a cleavable linker. Exemplary cleavable linkers usefully included in the instant high-efficiency conjugation moiety are known in the art. See, e.g., Leriche et al. (2012) Bioorg. Med. Chem. 20:571-582 (doi:10.1016/j.bmc.2011.07.048). Inclusion of a cleavable linker in the high-efficiency conjugation moiety allows for the selective cleavage of the bridging antigen from the oligonucleotide probe in the instant hybridization reagents. Such selective cleavage may be advantageous in some hybridization assay methods, for example where release of a bridging antigen and its associated secondary antibody from a sample surface is desired.

In other embodiments, the high-efficiency conjugation moiety is a non-covalent conjugation moiety. Non-limiting examples of a non-covalent conjugation moiety include an oligonucleotide hybridization pair or a protein-ligand binding pair. In specific embodiments, the protein-ligand binding pair is an avidin-biotin pair, a streptavidin-biotin pair, or another protein-biotin binding pair (see generally Avidin-Biotin Technology, Meth. Enzymol. (1990) volume 184, Academic Press; Avidin-Biotin Interactions: Methods and Applications (2008) McMahon, ed., Humana; Molecular Probes® Handbook, Chapter 4 (2010)), an antibody-hapten binding pair (see generally Molecular Probes® Handbook, Chapter 4 (2010)), an S-peptide tag-S-protein binding pair (Kim and Raines (1993) Protein Sci. 2:348-56), or any other high-affinity peptide-peptide or peptide-protein binding pair. Such high-affinity non-covalent conjugation moieties are well known in the art. Reactive versions of the respective conjugating pairs, for example thiol-reactive or amino-reactive versions, are also well known in the art. These conjugating reagents may be used to modify the respective oligonucleotide probe and bridging antigen. The modified oligonucleotide probe and bridging antigen may then be mixed in order to allow the complementary features, for example the oligonucleotide hybridization pair or the protein-ligand binding pair, to associate with one another and form a non-covalent high-efficiency conjugation moiety. All of the above-described covalent and non-covalent linking groups are capable of highly efficient association reactions and are thus well suited for use in generation of the instant hybridization reagents.

In some embodiments, the high-efficiency conjugation moiety is at least 50%, 80%, 90%, 93%, 95%, 97%, 98%, 99%, or even more efficient in coupling the oligonucleotide probe and the bridging antigen. In more specific embodiments, the high-efficiency conjugation moiety is at least 50%, 80%, 90%, 93%, 95%, 97%, 98%, 99%, or even more efficient at reactant concentrations of no more than 0.5 mg/mL. In some embodiments, the efficiencies are achieved at no more than 0.5 mg/mL, no more than 0.2 mg/mL, no more than 0.1 mg/mL, no more than 0.05 mg/mL, no more than 0.02 mg/mL, no more than 0.01 mg/mL, or even lower reactant concentrations.

In another aspect, the disclosure provides hybridization reagent compositions, also referred to as hybridization reagent panels, comprising a plurality of the above-described hybridization reagents. In embodiments, the composition comprises at least 3, 5, 10, 20, 30, 50, 100, or even more of the hybridization reagents. In some embodiments, the oligonucleotide probes of the included hybridization reagents are complementary to at least a segment of the genes encoding certain cellular markers, or the RNA expressed by those genes, and are thus capable of hybridizing to and detecting either the gene for the marker or expression of the gene for the marker. In specific embodiments, the cellular markers are at least ER and PR. In other specific embodiments, the cellular markers are at least HER2, ER, and PR or at least HER2, ER, and Ki67. In still other specific embodiments, the cellular markers are at least HER2, ER, PR, and Ki67. In yet still other specific embodiments, the cellular markers are at least Ki67, EGFR, and CK5. In even other specific embodiments, the cellular markers are at least Ki67, EGFR, CK5, and CK6, or are at least CK5, CK6, and Ki-67. In still other specific embodiments, the cellular markers are at least CK5, EGFR, p40, and Ki-67, or are at least IgA, complement 3c (C3c), collagen IV alpha chain 5 (COL4A5), and IgG. In some embodiments, the bridging antigens of the included immunoreagents are peptides.

In some embodiments, the immunoreagent compositions of the instant disclosure are specific for cellular markers on immune cells, for example, CD3, CD4, CD8, CD20, CD68, and/or FoxP3, in any combination, and any of the cellular markers listed above. In some embodiments, the immunoreagent compositions are specific for markers relating to checkpoint pathways, such as, for example, CTLA-4, CD152, PD-1, PD-L1, and the like.

Antigen-coupled immunoreagents comprising a primary antibody coupled to a bridging antigen have been disclosed previously in U.S. patent application Ser. No. 15/017,626 and PCT International Application No. PCT/US16/16913, both filed on Feb. 6, 2016, the disclosures of which are incorporated herein by reference in their entireties for all purposes. The methods exemplified in those disclosures for making and using bridging antigen-linked immunoreagents can be readily adapted in the synthesis and use of the instant hybridization reagents, as would be understood by those of ordinary skill in the art.

### Detectable Antibodies

As noted above, the bridging antigens of the instant hybridization reagents are recognizable by detectable antibodies. In order to increase sensitivity and decrease background in hybridization assays using the instant hybridization reagents, it is generally desirable to maximize the affinity and/or specificity of each detectable antibody for its corresponding bridging antigen. As is understood by those of ordinary skill in the art, affinities of antibodies for antigens are typically assessed using an equilibrium parameter, the dissociation constant or “K_{D}”. For a given concentration of antibody, the dissociation constant roughly corresponds to the concentration of antigen at which half the antibody is bound to an antigen and half the antibody is not bound to an antigen. Accordingly, a lower dissociation constant corresponds to a higher affinity of an antibody for the antigen.

The dissociation constant is also related to the ratio of the kinetic rate constants for dissociation and association of the antibody and the antigen. Dissociation constants may therefore be estimated either by equilibrium binding measurements or by kinetic measurements. Such approaches are well known in the art. For example, antibody-antigen binding parameters are routinely determined from the kinetic analysis of sensorgrams obtained using a Biacore surface plasmon resonance-based instrument (GE Healthcare, Little Chalfont, Buckinghamshire, UK), an Octet bio-layer interferometry system (Pall ForteBio Corp., Menlo Park, Calif.), or the like. See, for example, U.S. Patent Application Publication No. 2013/0331297 for a description of the determination of dissociation constants for a series of antibody clones and their corresponding peptide antigen binding partners.

Typical antibodies have equilibrium dissociation constants in the range from micromolar to high nanomolar (i.e., 10⁻⁶ M to 10⁻⁸ M). High affinity antibodies generally have equilibrium dissociation constants in the lower nanomolar to high picomolar range (i.e., 10⁻⁸ M to 10⁻¹⁰ M). Very high affinity antibodies generally have equilibrium dissociation constants in the picomolar range (i.e., 10⁻¹⁰ M to 10⁻¹² M). Antibodies against peptides or other large molecules typically have higher affinities (lower K_{D}s) for their antigens than antibodies against small-molecule haptens, which may display dissociation constants in the micromolar range or even higher.

The secondary antibodies of the instant hybridization reagent compositions may be optimized in order to increase their affinity for antigen-coupled oligonucleotide probes. For example, U.S. Patent Application Publication No. 2013/0331297 discloses methods for identifying antibody clones with high affinities that may be suitably modified to generate the detectable antibodies utilized in the instant hybridization reagent compositions. In these methods, a short DNA fragment encoding a synthetic peptide is fused to the heavy chains of the gene pool encoding an antibody library of interest, and yeast cells are transformed to generate a yeast display antibody library. The yeast cells are screened with a high-speed fluorescence-activated cell sorter (FACS) to isolate high-affinity antibody clones with high specificity. Compared to other yeast display systems such as Aga2, this system has an added advantage that the transformed yeast cells secrete sufficient amounts of antibodies into the culture medium to allow the culture media of the individual yeast clones to be assayed directly to determine specificity and affinity of the expressed antibodies, without requiring the additional steps of cloning and antibody purification for identification of candidate clones with the desired specificity and affinity.

The above-described yeast display library system makes use of antibody libraries generated from immunized rabbits to produce rabbit monoclonal antibodies with high specificity and affinity, thus harnessing the superior ability of the rabbit immune system to generate antibodies against small haptens or peptides with the efficiency of yeast display to isolate antibody clones with superior affinity and specificity. Using this approach, a panel of rabbit monoclonal antibodies against small molecules, peptides, and proteins was generated with antibody affinities in the range of <0.01 to 0.8 nM. These affinities surpass the affinities of most monoclonal antibodies from rodents generated using traditional hybridoma technology. The approach also overcomes inherent issues of low fusion efficiency and poor stability encountered with rabbit hybridoma technology.

While the above-described yeast display library system is one approach for optimizing binding affinities of the secondary antibodies used in the instant hybridization reagent compositions, it should be understood that any suitable approach may be used to optimize the affinities without limitation. In some cases, suitable high-affinity antibodies may be available without optimization.

Accordingly, in some embodiments, the detectable antibody is specific for the bridging antigen with a dissociation constant of at most 100 nM, at most 30 nM, at most 10 nM, at most 3 nM, at most 1 nM, at most 0.3 nM, at most 0.1 nM, at most 0.03 nM, at most 0.01 nM, at most 0.003 nM, or even lower. In more specific embodiments, the detectable antibody is specific for the bridging antigen with a dissociation constant of at most 1 nM, at most 0.3 nM, at most 0.1 nM, at most 0.03 nM, at most 0.01 nM, at most 0.003 nM, or even lower. In even more specific embodiments, the detectable antibody is specific for the bridging antigen with a dissociation constant of at most 100 pM, at most 30 pM, at most 10 pM, at most 3 pM, or even lower.

The antibody of the instant hybridization reagent compositions is preferably a detectable antibody, and in embodiments it therefore comprises a detectable label. As would be understood by those of ordinary skill in the art, the detectable label of the detectable antibody should be capable of suitable attachment to the antibody, and the attachment should be carried out without significantly impairing the interaction of the antibody with the bridging antigen.

In some embodiments, the detectable label may be directly detectable, such that it may be detected without the need for any additional components. For example, a directly detectable label may be a fluorescent dye, a biofluorescent protein, such as, for example, a phycoerythrin, an allophycocyanin, a peridinin chlorophyll protein complex (“PerCP”), a green fluorescent protein (“GFP”) or a derivative thereof (for example, a red fluorescent protein, a cyan fluorescent protein, or a blue fluorescent protein), luciferase (e.g., firefly luciferase, renilla luciferase, genetically modified luciferase, or click beetle luciferase), or coral-derived cyan and red fluorescent proteins (as well as variants of the red fluorescent protein derived from coral, such as the yellow, orange, and far-red variants), a luminescent species, including a chemiluminescent species, an electrochemiluminescent species, or a bioluminescent species, a phosphorescent species, a radioactive substance, a nanoparticle, a SERS nanoparticle, a quantum dot or other fluorescent crystalline nanoparticle, a diffracting particle, a Raman particle, a metal particle, including a chelated metal, a magnetic particle, a microsphere, an RFID tag, a microbarcode particle, or a combination of these labels.

In other embodiments, the detectable label may be indirectly detectable, such that it may require the employment of one or more additional components for detection. For example, an indirectly detectable label may be an enzyme that effects a color change in a suitable substrate, as well as other molecules that may be specifically recognized by another substance carrying a label or that may react with a substance carrying a label. Non-limiting examples of suitable indirectly detectable labels include enzymes such as a peroxidase, an alkaline phosphatase, a glucose oxidase, and the like. In specific embodiments, the peroxidase is a horseradish peroxidase or a soybean peroxidase. Other examples of indirectly detectable labels include haptens such as, for example, a small molecule or a peptide. Non-limiting exemplary haptens include nitrophenyl, dinitrophenyl, digoxygenin, biotin, a Myc tag, a FLAG tag, an HA tag, an S tag, a Streptag, a His tag, a V5 tag, a ReAsh tag, a FlAsh tag, a biotinylation tag, an Sfp tag, or another chemical or peptide tag.

In specific embodiments, the detectable label is a fluorescent dye. Non-limiting examples of suitable fluorescent dyes may be found in the catalogues of Life Technologies/Molecular Probes (Eugene, Oreg.) and Thermo Scientific Pierce Protein Research Products (Rockford, Ill.), which are incorporated by reference herein in their entireties. Exemplary dyes include fluorescein, rhodamine, and other xanthene dye derivatives, cyanine dyes and their derivatives, naphthalene dyes and their derivatives, coumarin dyes and their derivatives, oxadiazole dyes and their derivatives, anthracene dyes and their derivatives, pyrene dyes and their derivatives, and BODIPY dyes and their derivatives. Preferred fluorescent dyes include the DyLight fluorophore family, available from Thermo Scientific Pierce Protein Research Products.

In some embodiments, the detectable label may not be attached directly to the secondary antibody, but may be attached to a polymer or other suitable carrier intermediate that allows larger numbers of detectable labels to be attached to the secondary antibody than could normally be bound.

In specific embodiments, the detectable label is an oligonucleotide barcode tag, for example the barcode tags disclosed in PCT International Patent Publication No. WO2012/071428A2, the disclosure of which is incorporated herein by reference in its entirety. Such detectable labels are particularly advantageous in hybridization assays involving the isolation and/or sorting of targeted samples, for example in flow cytometry-based multiplexed assays, and the like. These labels are also advantageous in hybridization assays where the levels of target nucleic acid in a sample are low, and extreme sensitivity of detection is required.

In some embodiments, the detectable antibodies of the instant disclosure may comprise multiple detectable labels. In these embodiments, the plurality of detectable labels associated with a given secondary antibody may be multiple copies of the same label or may be a combination of different labels that result in a suitable detectable signal.

In some hybridization reagent composition embodiments, it may be advantageous for purposes of increasing the signal output from the composition to attach one or more detectable labels to the bridging antigen itself. The detectable labels usefully attached to the bridging antigen can be any of the above-described detectable labels. Such detectable labels should ideally overlap in detectability with the detectable label of the secondary antibody, so that the signals from an oligonucleotide probe-bridging antigen and secondary antibody pair will be additive. Furthermore, the attachment of a detectable label to a bridging antigen should ideally not significantly affect the binding of the secondary antibody to the bridging antigen. Likewise, the binding of the secondary antibody to the bridging antigen should ideally not significantly affect the detectability of the detectable label.

In preferred embodiments, the detectable label of the bridging antigen is a fluorophore. In more preferred embodiments, the detectable label of the bridging antigen and the detectable label of the secondary antibody are both fluorophores. In other preferred embodiments, the detectable label of the bridging antigen and the detectable label of the secondary antibody are both detectable by fluorescence at the same wavelength. In still other preferred embodiments, the detectable label of the bridging antigen and the detectable label of the secondary antibody are the same.

### Hybridization Reagent Composition Pairs

As mentioned above, the instant disclosure provides in some aspects hybridization reagent compositions comprising an oligonucleotide probe coupled to a bridging antigen and a detectable antibody specific for the bridging antigen. In these compositions, the detectable antibody and the antigen-conjugated oligonucleotide probe are paired due to the high affinity of the secondary antibody for the bridging antigen. It is understood that the paired composition will form whenever the separate components of the composition are mixed together in aqueous solution, for example whenever the reagents are used together in a hybridization assay.

Hybridization reagents comprising an oligonucleotide probe and coupled bridging antigen are described in detail above, as are detectable antibodies suitable for use in the instant hybridization reagent pairs. As would be understood by those of ordinary skill in the art, a composition comprising these components finds utility in the practice of hybridization assays, including CISH, FISH, and the like, alone or in combination with other diagnostic assays, such as IHC, cytometry, flow cytometry, such as fluorescence-activated cell sorting, microscopic imaging, pretargeting imaging, and other types of in vivo tumor and tissue imaging, high content screening (HCS), immunocytochemistry (ICC), immunomagnetic cellular depletion, immunomagnetic cell capture, sandwich assays, general affinity assays, enzyme immuno-assay (EIA), enzyme linked immuno-assay (ELISA), ELISpot, mass cytometry (CyTOF), arrays including microsphere arrays, multiplexed microsphere array, microarray, antibody array, cellular array, solution phase capture, lateral flow assays, chemiluminescence detection, infrared detection, blotting methods, including Western blots, Southwestern blot, dot blot, tissue blot, and the like, or combinations thereof.

### Multiplexed Hybridization Reagent Pairs

According to another aspect, the instant disclosure provides hybridization reagent compositions comprising a plurality of oligonucleotide probes coupled to a plurality of bridging antigens and a plurality of detectable antibodies. Each bridging antigen in these compositions is coupled to a different oligonucleotide probe, and at least one detectable antibody binds to each bridging antigen with high affinity. The plurality of antigen-coupled oligonucleotide probes and detectable antibodies in these compositions may be any of the hybridization reagent composition pairs described in the previous section.

In specific embodiments, the composition comprises at least three hybridization reagent composition pairs. In more specific embodiments, the composition comprises at least five hybridization reagent composition pairs. In still more specific embodiments, the composition comprises at least ten hybridization reagent composition pairs. In even more specific embodiments, the composition comprises at least 20, 30, 50, 100, or even more hybridization reagent composition pairs.

### Hybridization Reagent Panels

The hybridization reagents described above can be combined in pre-defined groups to create diagnostic panels for use in identifying specific genomic loci or chromosomal patterns or in monitoring the expression of specific combinations of genetic markers in certain tissues of interest, in particular in diseased tissues of interest such as in tumor tissues. Such panels are of use in diagnostic assays to identify such diseased tissues and are further of use as companion diagnostics, where the panels are used to monitor nucleic acid markers in the diseased tissues over time during the course of a particular treatment regimen. Such companion diagnostics provide for the timely and reliable assessment of the effectiveness of the treatment regimen and may further allow treatment dosages and frequency to be optimized for a particular patient. As is known in the art, the monitoring of target tissues using current in situ hybridization techniques may be limited by the number of oligonucleotide probes per tissue section or may require the staining of tissue sections separately or sequentially with different oligonucleotide probes. In contrast, the hybridization reagent panels disclosed herein allow high levels of multiplexing, such that the staining of a tissue or other sample of interest can be performed simultaneously with large numbers of oligonucleotide probes in single tissue sections or other samples.

According to this aspect, the invention therefore provides hybridization reagent comprising at least three hybridization reagents of the instant disclosure. In specific embodiments, the hybridization reagent comprise at least five, at least at least ten, at least 15, at least 20, at least 30, or even more hybridization reagents of the instant disclosure, as described in detail above.

Of particular interest is the use of the instant hybridization reagent panels to profile tissue samples in patients being treated using immunotherapeutic regimens, for example in the treatment of autoimmune diseases and cancer. Recent advances in the blockade of checkpoint pathways, for example using antibodies targeting the cytotoxic T lymphocyte-associated antigen-4 (CTLA-4, CD152) (e.g., ipilimumab) or antibodies targeting the programed death receptors or their ligands (PD-1 or PD-L1) (e.g., pembrolizumab, nivolumab, pidilizumab, and the like), have been shown to be especially effective. See, e.g., Adams et al. (2015) Nature Rev. Drug Discov. 14:603-22; Mahoney et al. (2015) Nature Rev. Drug Discov. 14:561-84; Shin et al. (2015) Curr. Opin. Immunol. 33:23-35.

Other recently approved anticancer agents target other cell-surface proteins or gene products that are upregulated or amplified in tumors and other diseases (see, e.g., rituximab against CD20 in lymphoma cells, trastuzumab against HER2/neu in breast cancer cells, cetuximab against EGFR in various tumor cells, bevacizumab against VEGF in a variety of cancer cells and in the eye, and denosumab against osteoclasts in bone). The profiling of tissue samples from patients being treated with these agents is thus also of great current interest in clinical medicine.

Likewise, tissue samples obtained from patients either prior to or during treatment with anticancer agents may also benefit from molecular profiling. For example, patients being treated with imatinib, lenalidomide, pemetrexed, bortezomib, leuprorelin, abiraterone acetate, ibrutinib, capecitabine, erlotinib, everolimus, sirolimus, nilotinib, sunitinib, sorafenib, and the like can be advantageously monitored by the profiling of tissues, in particular diseased tissues, using the instant immunoreagent panels.

Methods and systems for the molecular profiling of tissues, including the analysis of immune modulators, and the use of those profiles to assess and monitor disease treatments have also been reported. See, e.g., U.S. Pat. Nos. 8,700,335 B2; 8,768,629 B2; 8,831,890 B2; 8,880,350 B2; 8,914,239 B2; 9,053,224 B2; 9,058,418 B2; 9,064,045 B2; 9,092,392 B2; PCT International Patent Publication No. WO 2015/116868. Such approaches are advantageously performed using suitable panels of the instant hybridization reagents.

Exemplary panels identify the expression of combinations of tumor cell, immune cell, and various disease-related genetic markers, including the following markers:

4-1BB, AFP, ALK1, Amyloid A, Amyloid P, Androgen Receptor, Annexin A1, ASMA, BCA225, BCL-1, BCL-2, BCL-6, BerEP4, Beta-Catenin, Beta-HCG, BG-8, BOB-1, CA19-9, CA125, Calcitonin, Caldesmon, Calponin-1, Calretinin, CAM 5.2, CD1a, CD2, CD3, CD4, CD5, CD7, CD8, CD10, CD15, CD19, CD20, CD21, CD22, CD23, CD25, CD30, CD31, CD33, CD34, CD38, CD42b, CD43, CD45 LCA, CD45RO, CD56, CD57, CD61, CD68, CD79a, CD99, CD117, CD138, CD163, CDX2, CEA, Chromogranin A, CMV, c-kit, c-MET, c-MYC, Collagen Type IV, Complement 3c (C3c), COX-2, CXCR5, CK1, CK5, CK6, CK7, CK8, CK14, CK18, CK17, CK19, CK20, CK903, CK AE1, CK AE1/AE3, D2-40, Desmin, DOG-1, E-Cadherin, EGFR, EMA, ER, ERCC1, Factor VIII-RA, Factor XIIIa, Fascin, FoxP1, FoxP3, Galectin-3, GATA-3, GCDFP-15, GCET1, GFAP, Glycophorin A, Glypican 3, Granzyme B, HBME-1, Helicobacter Pylori, Hemoglobin A, Hep Par 1, HER-2, HHV-8, HMB-45, HSV 1/11, ICOS, IFNgamma, IgA, IgD, IgG, IgM, IL17, IL4, Inhibin, iNOS, Kappa Ig Light Chain, Ki-67, LAG-3, Lambda Ig Light Chain, Lysozyme, Mammaglobin A, MART-1/Melan A, Mast Cell Tryptase, MLH1, MOC-31, MPO, MSA, MSH2, MSH6, MUC1, MUC2, MUM1, MyoD1, Myogenin, Myoglobin, Napsin A, Nestin, NSE, Oct-2, OX40, OX40L, p16, p21, p27, p40, p53, p63, p504s, PAX-5, PAX-8, PD-1, PD-L1, PHH3, PIN-4, PLAP, PMS2, Pneumocystis jiroveci (carinii), PgR, PSA, PSAP, RCC, S-100, SMA, SMM, Smoothelin, SOX10, SOX11, Surfactant Apoprotein A, Synaptophysin, TAG 72, TdT, Thrombomodulin, Thyroglobulin, TIA-1, TIM3, TRAcP, TTF-1, Tyrosinase, Uroplakin, VEGFR-2, Villin, Vimentin, WT-1, and the like.

Preferably, the panels identify the expression of one or more of the following markers: CD4, CD8, CD20, CD68, PD-1, PD-L1, FoxP3, SOX10, Granzyme B, CD3, CD163, IL17, IL4, IFNgamma, CXCR5, FoxP1, LAG-3, TIM3, CD34, OX40, OX40L, ICOS, and 4-1BB.

The panels are provided either in kit form or as a group of the different hybridization reagents provided separately for use in the methods for in situ hybridization described in detail below. In particular, the panels are used in multiplexed methods, where samples are reacted with multiple hybridization reagents for simultaneous detection. The hybridization reagents are any of the above-described hybridization reagents, in particular those comprising an oligonucleotide probe complementary to any of the above-defined target genetic markers, and a bridging antigen, wherein the oligonucleotide probe and the bridging antigen are coupled, and wherein the bridging antigen is recognized by a detectable antibody with high affinity.

In specific embodiments, the panels identify the expression of the following exemplary combinations of genetic markers:
CD4, CD8, CD68, and PD-L1;
CD4, CD8, FoxP3, and CD68 (for any solid tumor);
CD8, CD68, PD-L1, plus tumor associated marker (for head and neck and pancreatic tumors);
SOX10, CD8, PD-1, and PD-L1 (for melanoma);
CD4, CD8, CD20, and cytokeratin (for breast cancer TIL);
CD8, CD34, FoxP3, and PD-L1 (for melanoma immunology);
CD8, CD34, PD-L1, and FoxP1 (for cancer immunology);
CD3, PD1, LAG-3, and TIM3 (for T cell exhaustion);
CD4 and FoxP3 (for Treg);
CD4 and IL17 (for Th17);
CD8 and Granzyme B (for activated CD8);
CD4 and CXCR5 (for TFh);
CD4 and IL4 (for Th2);
CD4 and IFNg (for Th1);
CD4, CD8, CD3, and CD20 (for general lymphocytes);
CD4, CD8, CD68, and CD20 (for lymphocytes and macrophages);
CD4, FoxP3, CD8, and CD20 (for Treg and lymphocytes);
CD4, FoxP3, CD8, and Granzyme B (for Treg and Act CTL);
CD68 (for macrophages);
CD68 and CD163 (for M2 macrophages);
CD20 (for B cells); and
OX40, OX40L, ICOS, and 41BB (for other molecules of interest)

### Methods of In Situ Hybridization

In another aspect, the instant disclosure provides methods of in situ hybridization, comprising reacting a hybridization reagent with a target nucleic acid, reacting a detectable antibody with the hybridization reagent, wherein the detectable antibody binds to the bridging antigen of the hybridization reagent with high affinity, and detecting the bound detectable antibody. The hybridization reagent and detectable antibody in these methods may usefully be any of the above-described hybridization reagents and detectable antibodies, in any suitable combination.

In embodiments, the method of detection is an in situ hybridization method. As described above, in situ hybridization is widely used technique that is applied frequently to the diagnosis of abnormal cells, such as tumor cells. The expression of specific genetic markers is characteristic of a particular tumor cell, for example a breast cancer cell. In situ hybridization assays are also frequently used to understand the distribution and localization of chromosomal markers and differentially expressed nucleic acid markers in different parts of a biological tissue.

In specific embodiments, the target nucleic acid is present within a tissue section. Detection of nucleic acids within tissue sections is well understood by those of skill in the clinical pathology arts. Such approaches have even been used to identify the total copy number of mRNAs in intact cells and tissues at the single-molecule/single-cell level. See, e.g., Raj et al. (2008) Nature Methods 5:877; Larson et al. (2009) Trends Cell Biol. 19:630; www.singlemoleculefish.com. It should be understood that solid tissue samples, typically following a fixation process, can be sectioned in order to expose one or more target nucleic acids of interest on the surface of the sample. The analysis of consecutive tissue sections, i.e., sections that had been adjacent, or nearly adjacent, to one another in the original tissue sample, enables the recreation of a three-dimensional model of the original tissue sample, or the increased capability for multiplexing of target nucleic acids, as will be described in more detail below. In preferred embodiments, the first target nucleic acid is a target nucleic acid within a tissue section of a tumor sample.

In other specific embodiments, the nucleic acid detected by the method is in or on a cell. Such detection is well understood, for example, by those of skill in the art of cytometry. In some embodiments, the nucleic acid may be on the surface of a cell. In other embodiments, the nucleic acid may be in the cytoplasm of a cell. In still other embodiments, the nucleic acid may be in the nucleus of a cell. In some embodiments, the nucleic acid may be in more than one location in the cell.

The tissue analyzed according to the above methods may be any suitable tissue sample. For example, in some embodiments, the tissue may be connective tissue, muscle tissue, nervous tissue, or epithelial tissue. Likewise, the tissue analyzed may be obtained from any organ of interest. Non-limiting examples of suitable tissues include breast, colon, ovary, skin, pancreas, prostate, liver, kidney, heart, lymphatic system, stomach, brain, lung, and blood.

In some embodiments, the detecting step is a fluorescence detection step. Suitable fluorescence detection labels are described in detail above.

In some embodiments, the method of detection further comprises the step of sorting cells that have bound the detectable antibody. Cell sorting is a well understood technique within the art of flow cytometry. Exemplary flow cytometry methods of detection are provided, for example, in Practical Flow Cytometry, 4^{th} ed., Shapiro, Wiley-Liss, 2003; Handbook of Flow Cytometry Methods, Robinson, ed., Wiley-Liss, 1993; and Flow Cytometry in Clinical Diagnosis, 4^{th} ed., Carey et al., eds, ASCP Press, 2007. The use of hydrazone-linked antibody-oligonucleotide conjugates in quantitative multiplexed immunologic assays, in particular, in quantitative flow cytometric assays, is described in PCT International Publication No. WO 2013/188756 and in Flor et al. (2013) Chembiochem. 15:267-75.

In some embodiments, the method of hybridization assay comprises reacting additional hybridization reagents with additional target nucleic acids in multiplexed assays, wherein the additional hybridization reagents are any of the above-defined hybridization reagents complementary to the additional target nucleic acids, reacting the additional hybridization reagents with additional detectable antibodies, wherein the additional detectable antibodies bind to the bridging antigens of the additional hybridization reagents with high affinity, and detecting the bound detectable additional antibodies. It should be understood that the order of reaction of the additional hybridization reagents and antibodies in the multiplexed methods may be varied in any suitable way in order to achieve desired results, as would be understood by those of ordinary skill in the art. In some embodiments, all of the different hybridization reagents may be added simultaneously to a target sample containing multiple target nucleic acids. In other embodiments, the different hybridization reagents may be added sequentially, in any order. Likewise with the secondary antibodies, which may be added either simultaneously or sequentially, in any order. In the multiplexed assays, the methods may detect 2, 3, 5, 10, 20, 30, 50, 100, or even more different target nucleic acids in a single assay. As described in detail above, the ability of the instant hybridization reagents to be used in such higher-level multiplexed hybridization assays is a major advantage of the instant hybridization reagents. In particular, these hybridization reagents enable hybridization assays with exquisite sensitivity, selectivity, and extremely low levels of background signal.

In some embodiments, the instant methods of hybridization assay comprise the analysis of adjacent or nearly adjacent sections of a fixed tissue sample in order to increase the level of multiplexing of detectable nucleic acids possible for a given tissue sample or to recreate a three-dimensional image of the sample. For example, in some embodiments the methods further comprise the step of reacting a second hybridization reagent with a second target nucleic acid on a second sample. In some of these methods, the first sample and the second sample may be serial sections of a tissue sample (i.e., sections that are adjacent, or nearly adjacent, to one another in the sample), and the second hybridization reagent is any of the above hybridization reagents complementary to the second nucleic acid. The methods further comprise the step of reacting a second detectable antibody with the second hybridization reagent, wherein the second detectable antibody is specific for the bridging antigen of the second hybridization reagent with high affinity, and the step of detecting the second detectable antibody that is associated with the bridging antigen of the second hybridization reagent.

It will be understood that the hybridization assay of serial sections of a given tissue sample provides for the greatly increased multiplexing of nucleic acid detection in view of current hardware and software limitations. For example, although the hybridization reagents and methods described herein in principle allow unlimited multiplexing due to the unlimited variation in bridging antigens and secondary antibodies, such assays are nevertheless limited by the number of fluorescent dyes that can currently be distinguished simultaneously on a single tissue section with available detection devices. Serial sections of the same tissue sample can, however, be stained with different panels of oligonucleotide probes to identify different sets of target nucleic acids by the reuse of the same panel of detectable labels, for example fluorescent labels, on the different sections. The detectable labels may be attached to the same set of secondary antibodies used in labeling the first sample section, in which case the second panel of oligonucleotide probes would be labeled with the same set of bridging antigens as used with the first panel of oligonucleotide probes. Alternatively and optionally, the detectable labels may be attached to a different set of secondary antibodies used in labeling the first sample section, in which case the second panel of oligonucleotide probes would be labeled with a different set of bridging antigens than were used with the first panel of oligonucleotide probes.

It will also be understood that the hybridization assay of serial sections of a given tissue sample enable the analysis of target tissue nucleic acids in a third dimension, thus providing further information regarding the overall structure of the sample tissue, for example by tomographic techniques. In some embodiments, the first sample and the second sample may not be serial sections of the sample but may instead be separated in space within the original tissue, thus providing still further information about the relative spatial positioning of target nucleic acids in the third dimension. Those of ordinary skill in the art will understand the utility of serial section images in the reconstruction of three-dimensional tissue structures.

In some embodiments, a plurality of target nucleic acids are detected on each of the samples. In specific embodiments, at least two target nucleic acids, at least three target nucleic acids, at least five target nucleic acids, at least ten target nucleic acids, at least 15 target nucleic acids, at least 25 target nucleic acids, or even more target nucleic acids are detected on each of the samples. In some embodiments, one or more target nucleic acids are detected on at least three samples, at least four samples, at least five samples, at least ten samples, at least 15 samples, at least 25 samples, or even more.

In another aspect, the instant disclosure provides methods of hybridization assay where a plurality of target nucleic acids in a sample are labeled by an initial treatment with oligonucleotide probes comprising bridging antigens and subsequent sequential treatments with reactive antibodies specific for the bridging antigens. Specifically, a sample comprising a first target nucleic acid and a second target nucleic acid is reacted with a first hybridization reagent complementary to the first target nucleic acid and a second hybridization reagent complementary to the second target nucleic acid, wherein the first hybridization reagent and the second hybridization reagent are any of the above-described hybridization reagents. The first hybridization reagent is reacted with a first reactive antibody, wherein the first reactive antibody binds to the bridging antigen of the first hybridization reagent with high affinity. The location of the first nucleic acid in the sample is then highlighted by reacting the first reactive antibody with a first detectable reagent, wherein the first detectable reagent is thereby bound to the sample in proximity to the first nucleic acid. The first reactive antibody is then selectively dissociated from the sample, and the second hybridization reagent is reacted with a second reactive antibody, wherein the second reactive antibody binds to the bridging antigen of the second hybridization reagent with high affinity. The location of the second nucleic acid in the sample is then highlighted by reacting the second reactive antibody with a second detectable reagent, wherein the second detectable reagent is thereby bound to the sample in proximity to the second nucleic acid. The first detectable reagent and the second detectable reagent are then detected, thus identifying the locations of the first target nucleic acid and the second target nucleic acid on the sample.

In specific embodiments of these methods, the first reactive antibody and the second reactive antibody each comprise an enzyme activity, more specifically a peroxidase activity such as a horse radish peroxidase activity. In other specific embodiments, either the first detectable reagent or the second detectable reagent comprises a tyramide, or each of the first detectable reagent and the second detectable reagent comprises a tyramide. In still other specific embodiments, either the first detectable reagent or the second detectable reagent comprises a fluorophore or a chromophore, or each of the first detectable reagent and the second detectable reagent comprises a fluorophore or a chromophore.

In preferred embodiments, the first reactive antibody is dissociated from the sample by a selective treatment. Specifically, the selective treatment may dissociate the first reactive antibody from the sample without dissociating the oligonucleotide probes from the sample. More specifically, the selective treatment may comprise treatment with a soluble bridging antigen. Such a treatment may involve the use of relatively high concentrations of the soluble bridging antigen, for example at least 1 μM, at least 10 μM, at least 100 μM, at least 1 mM, at least 10 mM, or even higher concentrations of the soluble bridging antigen, as would be understood by those of ordinary skill in the art.

It should also be understood that in the above methods, the steps of dissociating the reactive antibody from the sample, reacting an additional hybridization reagent with an additional target nucleic acid on the sample, reacting an additional reactive antibody with the additional hybridization reagent, and reacting the additional reactive antibody with an additional detectable reagent, so that the additional detectable reagent is bound to the sample in proximity to the additional target nucleic acid, may be repeated as many times as necessary in order to detect the locations of as many target nucleic acids on the sample as desired. In some embodiments, the steps are repeated so as to detect the location of at least three target nucleic acids, at least four target nucleic acids, at least five target nucleic acids, at least ten target nucleic acids, or even more target nucleic acids on the sample.

It should also be understood that the order of the steps used in these assay methods may depend on the particular reaction conditions used, and that additional reaction steps may also be necessary to complete the assays in some cases. For example, if a non-selective method is used to dissociate the reactive antibody from the sample (e.g., heat, denaturation, etc.), it may be necessary to include additional reaction steps in the assays. Specifically, if the dissociation conditions also remove oligonucleotide probes from the sample, a further reaction with an additional hybridization reagent prior to reaction with an additional reactive antibody and an additional detectable reagent may be included in the process. In other words, the reaction of a new hybridization reagent with a new target nucleic acid will be included in the process for each target nucleic acid. In preferred embodiments, however, where the reactive antibodies are dissociated selectively, all of the desired hybridization reagents for reaction with all of the desired target nucleic acids may be added in an initial reaction step, and only the reactive antibodies are added in subsequent cycles. Use of selective treatments to dissociate reactive antibodies from the sample minimizes damage to the sample from harsh treatments and therefore improves outcomes from the assays.

The hybridization reagents of the instant disclosure may be usefully employed in a variety of in situ hybridization methods of detection, including without limitation chromogenic in situ hybridization (CISH), fluorescent in situ hybridization (FISH), and the like, alone or in combination, and optionally in combination with other diagnostic assays, such as microscopic imaging, pretargeting imaging, and other types of in vivo tumor and tissue imaging, high content screening (HCS), immunocytochemistry (ICC), immunomagnetic cellular depletion, immunomagnetic cell capture, sandwich assays, general affinity assays, enzyme immuno-assay (EIA), enzyme linked immuno-assay (ELISA), ELISpot, mass cytometry (CyTOF), arrays including microsphere arrays, multiplexed microsphere array, microarray, antibody array, cellular array, solution phase capture, lateral flow assays, chemiluminescence detection, infrared detection, blotting methods, including Western blots, Southwestern blot, dot blot, tissue blot, and the like, or combinations thereof. Each of these assays may benefit from the high level of multiplexing achieved using the instant hybridization reagents.

The above methods find use in research and clinical settings, without limitation. They may be used for diagnostic purposes, including predictive screening and in other types of prognostic assays, for example in a diagnostic laboratory setting or for point of care testing. The instant multiplexed hybridization technology is also well-suited for use in high-throughput screens.

Immunohistochemical staining, including multiplexed immunohistochemical staining, of tissue sections with bridging antigen-labeled primary antibodies and detectable anti-bridging antigen secondary antibodies is exemplified in U.S. patent application Ser. No. 15/017,626 and PCT International Application No. PCT/US16/16913. Such techniques can be adapted for use in in situ hybridization assays using the hybridization reagent compositions of the instant disclosure, as would be understood by those of ordinary skill in the art.

### Methods of Preparation

In another aspect, the instant disclosure provides novel methods of preparing antigen-coupled hybridization reagents such as the hybridization reagents described above. In some embodiments, the methods comprise the step of coupling an oligonucleotide probe to a bridging antigen using a chemical coupling reaction. In specific embodiments, the oligonucleotide probe and the bridging antigen are coupled by a high-efficiency conjugation moiety. In some embodiments the methods comprise the steps of modifying an oligonucleotide probe with a first conjugating reagent, modifying a bridging antigen with a second conjugating reagent, and reacting the modified oligonucleotide probe with the modified bridging antigen to generate an antigen-coupled hybridization reagent. In specific embodiments, the first conjugating reagent and the second conjugating reagent associate with one another at high efficiency.

By high-efficiency, it is meant that the efficiency of conversion of oligonucleotide probe to antigen-coupled oligonucleotide probe is at least 50%, 70%, 90%, 95%, or 99% complete under the conditions of the conjugation reaction. In some embodiments, these efficiencies are achieved at no more than 0.5 mg/mL, no more than 0.2 mg/mL, no more than 0.1 mg/mL, no more than 0.05 mg/mL, no more than 0.02 mg/mL, no more than 0.01 mg/mL, or even lower protein concentrations.

The oligonucleotide probes and bridging antigens usefully employed in the methods of preparation include any of the oligonucleotide probes and bridging antigens described above. The first and second conjugating reagents are chosen according to the desired outcomes. In particular, high-efficiency conjugating reagents capable of specific and selective reaction with amino or thiol groups are of particular utility in the modification of amino- or thiol-modified oligonucleotide probes and amino- or thiol-containing bridging antigens. In addition, the first and second conjugating reagents are chosen for their ability to associate with one another at high efficiency, and thus to create the high-efficiency conjugation moiety in some of the above-described antigen-coupled hybridization reagents.

As described above, the resulting conjugation moiety may be a covalent moiety or a non-covalent moiety, and the first and second conjugating reagents used to prepare the modified oligonucleotide probes and modified bridging antigens are chosen accordingly. For example, in the case of a non-covalent conjugation moiety, the first conjugating reagent preferably comprises a selectively reactive group to attach the reagent to particular reactive residues of the oligonucleotide and a first component of the conjugation pair. Likewise, the second conjugating reagent preferably comprises a selectively reactive group to attach the reagent to particular reactive residues of the bridging antigen and a second component of the conjugation pair. The first and second components of the conjugation pairs are able to associate with one another non-covalently at high efficiency and thus to generate the antigen-coupled hybridization reagent.

As previously described, examples of non-covalent conjugation moieties include oligonucleotide hybridization pairs and protein-ligand binding pairs. In the case of an oligonucleotide hybridization pair, for example, the oligonucleotide probe would be reacted with a first conjugating reagent that comprises one member of the hybridization pair, and the bridging antigen would be reacted with a second conjugating reagent that comprises the second member of the hybridization pair. The modified oligonucleotide probe and the modified bridging antigen can thus be mixed with one another, and the association of the two members of the hybridization pair generates the high-efficiency conjugation moiety.

Likewise, when a protein-ligand binding pair is used to generate the non-covalent conjugation moiety of the antigen-coupled hybridization reagent, the oligonucleotide is reacted with a first conjugating reagent that comprises one or the other of the protein-ligand pair, and the bridging antigen is reacted with a second conjugating reagent that comprises the complementary member of the protein-ligand pair. The so-modified oligonucleotide and bridging antigen are then mixed with one another to generate the high-efficiency conjugation moiety.

As was described in detail above, examples of high-efficiency covalent conjugation moieties include hydrazones, oximes, other Schiff bases, and the products of any of the various click reactions. Exemplary hydrazino, oxyamino, and carbonyl conjugating reagents for use in forming the high-efficiency conjugation moieties are illustrated in U.S. Pat. No. 7,102,024 and can be adapted for use in the instant reaction methods. As described therein, the hydrazine moiety may be an aliphatic, aromatic, or heteroaromatic hydrazine, semicarbazide, carbazide, hydrazide, thiosemicarbazide, thiocarbazide, carbonic acid dihydrazine, or hydrazine carboxylate. The carbonyl moiety may be any carbonyl-containing group capable of forming a hydrazine or oxime linkage with one or more of the above-described hydrazine or oxyamino moieties. Preferred carbonyl moieties include aldehydes and ketones. Activated versions of some of these reagents, for use as conjugating reagents in the instant methods, are available commercially, for example from Solulink, Inc. (San Diego, Calif.) and Jena Bioscience GmbH (Jena, Germany). In some embodiments, the reagents may be incorporated into the oligonucleotide or the bridging antigen during the synthesis of the oligonucleotide or the bridging antigen, for example during a solid phase synthesis reaction.

The incorporation of hydrazine, oxyamino, and carbonyl-based monomers into oligonucleotides for use in immobilization and other conjugation reactions is described in U.S. Pat. Nos. 6,686,461; 7,173,125; and 7,999,098. Hydrazine-based and carbonyl-based bifunctional crosslinking reagents for use in the conjugation and immobilization of biomolecules is described in U.S. Pat. No. 6,800,728. The use of high-efficiency bisaryl-hydrazone linkers to form oligonucleotide conjugates in various detection assays and other applications is described in PCT International Publication No. WO 2012/071428. Each of the above references is hereby incorporated by reference herein in its entirety.

In some embodiments, the hybridization reagents of the instant disclosure are prepared using novel conjugating reagents and conditions. For example a thiol-reactive maleimido oxyamino (MOA) conjugating reagent useful in the preparation of antigen-coupled hybridization reagents may be prepared as shown in Scheme 1:

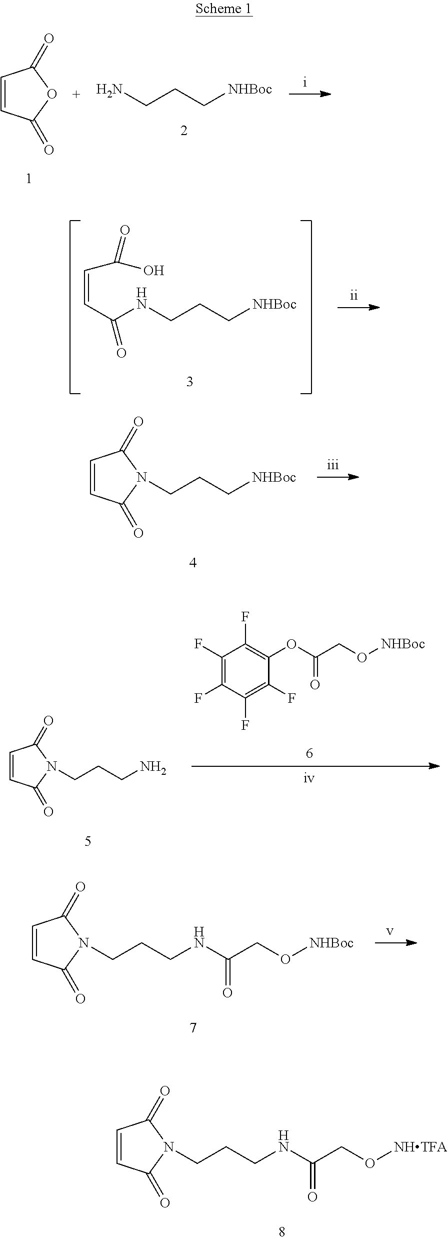

An amino-reactive oxyamino conjugating reagent (AOA) may be prepared as shown in Scheme 2:

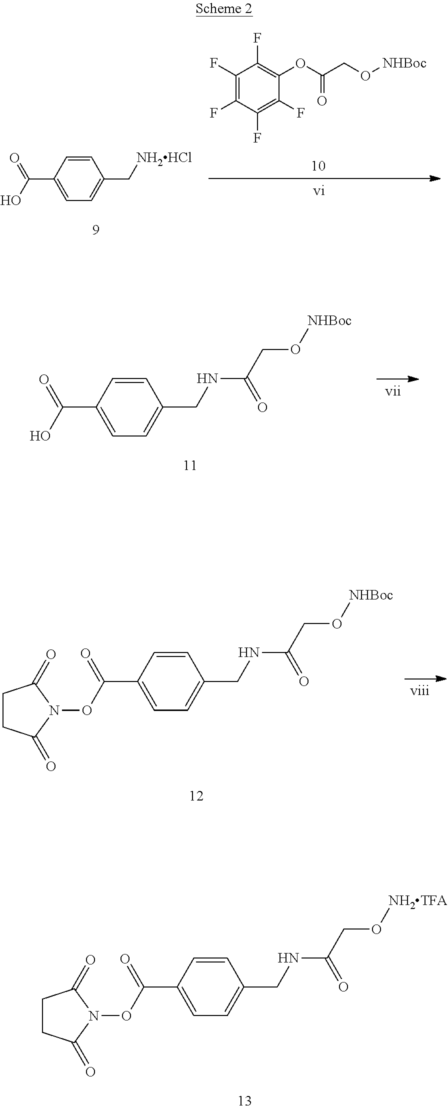

Alternative thiol-reactive and amino-reactive conjugating reagents may be prepared using variants of the above reaction schemes, as would be understood by those of ordinary skill in the art of synthetic chemistry. Such alternative reagents should be considered within the scope of the preparation methods disclosed herein.

Oligonucleotides and bridging antigens modified using one or another of the above oxyamino-containing reagents may usefully be reacted with a complementary oligonucleotide or bridging antigen that is itself modified with a carbonyl-containing reagent, for example, an aromatic aldehyde such as a formylbenzoate group. Alternative examples of such a conjugation reactions are shown in Schemes 3 and 4, where the R₁ and R₂ groups represent independently an oligonucleotide probe or a bridging antigen.

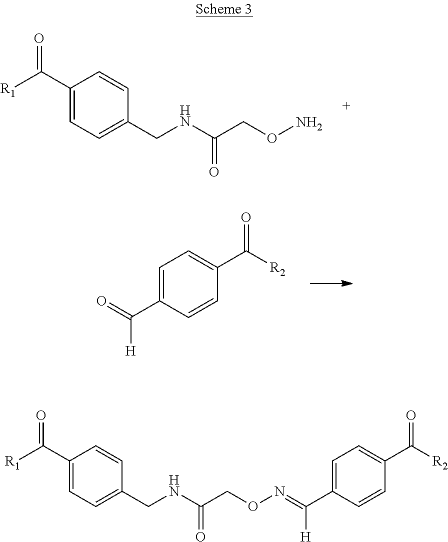

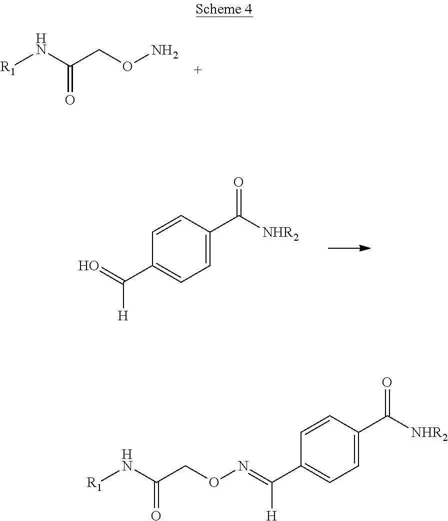

It should be understood that the relative orientation of the different members of the conjugation moiety-forming groups on the oligonucleotide probe and on the bridging antigen are generally not believed to be important, so long as the groups are able to react with one another to form the high-efficiency conjugation moiety. In other words, in the examples of Schemes 3 and 4, the R₁ group could be the oligonucleotide probe and the R₂ group could be the bridging antigen, or the R₁ group could be the bridging antigen and the R₂ group could be the oligonucleotide probe. The same is generally true for all of the above-described conjugating pairs, whether covalent or non-covalent.

The above-described conjugation methods provide several advantages over traditional crosslinking methods, for example methods using bifunctional crosslinking reagents. In particular, the reactions are specific, efficient, and stable. The specificity means that side reactions, such as homoconjugation reactions, do not occur, or occur at extremely low levels. The efficiency means that the reactions run to completion, or near completion, even at low reagent concentrations, thus generating products in at or near stoichiometric amounts. The stability of the conjugation moieties formed means that the resultant hybridization reagents can be used for a wide variety of purposes without concern that the conjugated products will dissociate during use. In some cases, the above conjugation methods allow the further advantage that the progress of the conjugation reaction may be monitored spectroscopically, since in some of the reactions a chromaphore is formed as the reaction occurs.

The synthesis and stabilities of hydrazone-linked adriamycin/monoclonal antibody conjugates are described in Kaneko et al. (1991) Bioconj. Chem. 2:133-41. The synthesis and protein-modifying properties of a series of aromatic hydrazides, hydrazines, and thiosemicarbazides are described in U.S. Pat. Nos. 5,206,370; 5,420,285; and 5,753,520. The generation of conjugationally-extended hydrazine compounds and fluorescent hydrazine compounds is described in U.S. Pat. No. 8,541,555.

Preparation of bridging antigen-labeled primary antibodies is exemplified in U.S. patent application Ser. No. 15/017,626 and PCT International Application No. PCT/US16/16913. Similar approaches can be used for the preparation of the instant hybridization reagents, as would be understood by those of ordinary skill in the art.

### Diagnostic Kits

In another aspect, the instant disclosure provides kits for use in hybridization assays for diagnostic or research purposes. The diagnostic kits comprise one or more hybridization reagents of the instant disclosure, together with instructions for use in a hybridization assay. In some embodiments, the kits further comprise a secondary antibody, for example a secondary antibody that is specific for the bridging antigen of the hybridization reagent at high affinity. Furthermore, it should be understood that the hybridization reagent included in the instant kits will typically comprise an oligonucleotide probe directed at a specific genetic marker, so that the kit may be used in hybridization assays to identify specific genomic loci or chromosomal patterns or to monitor the expression of one or more genetic markers in a tissue sample, in a suspension of cells, on another surface, or in another medium.

In further embodiments, the kits may comprise further components such as, for example, buffers of various compositions to enable usage of the kit for staining cells or tissues; and cellular counterstains to enable visualization of sample morphology. Kits may be provided in various formats and include some or all of the above listed components, or may include additional components not listed here.

Other aspects of the invention will be understood by reference to U.S. patent application Ser. No. 15/017,626 and PCT International Application No. PCT/US16/16913.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

What is claimed is:

1. A hybridization reagent composition comprising:
an oligonucleotide probe coupled to a bridging antigen; and
a detectable antibody;
wherein the detectable antibody is specific for the bridging antigen with high affinity.

2. The hybridization reagent composition of claim 1, wherein the bridging antigen is a peptide.

3. The hybridization reagent composition of claim 1, wherein the bridging antigen comprises a plurality of antigenic determinants.

4. The hybridization reagent composition of claim 3, wherein each antigenic determinant in the plurality of antigenic determinants is the same.

5. The hybridization reagent composition of claim 3, wherein the plurality of antigenic determinants comprises a linear repeating structure.

6. The hybridization reagent composition of claim 5, wherein the linear repeating structure is a linear repeating peptide structure.

7. The hybridization reagent composition of claim 3, wherein the plurality of antigenic determinants comprises at least three antigenic determinants.

8. The hybridization reagent composition of claim 3, wherein the bridging antigen comprises a branched structure.

9. The hybridization reagent composition of claim 1, wherein the bridging antigen is a peptide comprising a non-natural residue.

10. The hybridization reagent composition of claim 9, wherein the non-natural residue is a non-natural stereoisomer.

11. The hybridization reagent composition of claim 9, wherein the non-natural residue is a β-amino acid.

12. The hybridization reagent composition of claim 1, wherein the oligonucleotide probe and the bridging antigen are coupled by a chemical coupling reaction through a conjugation moiety.

13. The hybridization reagent composition of claim 12, wherein the oligonucleotide probe and the bridging antigen are coupled through a high-efficiency conjugation moiety.

14. The hybridization reagent composition of claim 13, wherein the high-efficiency conjugation moiety is a Schiff base.

15. The hybridization reagent composition of claim 14, wherein the Schiff base is a hydrazone or an oxime.

16. The hybridization reagent composition of claim 13, wherein the high-efficiency conjugation moiety is formed by a click reaction.

17. The hybridization reagent composition of claim 12, wherein the conjugation moiety comprises a cleavable linker.

18. The hybridization reagent composition of claim 1, wherein the oligonucleotide probe is complementary to at least a segment of a gene encoding a cellular marker or an RNA expressed by the gene.

19. The hybridization reagent composition of claim 18, wherein the cellular marker is selected from the group consisting of: 4-1BB, AFP, ALK1, Amyloid A, Amyloid P, Androgen Receptor, Annexin A1, ASMA, BCA225, BCL-1, BCL-2, BCL-6, BerEP4, Beta-Catenin, Beta-HCG, BG-8, BOB-1, CA19-9, CA125, Calcitonin, Caldesmon, Calponin-1, Calretinin, CAM 5.2, CD1a, CD2, CD3, CD4, CD5, CD7, CD8, CD10, CD15, CD19, CD20, CD21, CD22, CD23, CD25, CD30, CD31, CD33, CD34, CD38, CD42b, CD43, CD45 LCA, CD45RO, CD56, CD57, CD61, CD68, CD79a, CD99, CD117, CD138, CD163, CDX2, CEA, Chromogranin A, CMV, c-kit, c-MET, c-MYC, Collagen Type IV, Complement 3c (C3c), COX-2, CXCR5, CK1, CK5, CK6, CK7, CK8, CK14, CK18, CK17, CK19, CK20, CK903, CK AE1, CK AE1/AE3, D2-40, Desmin, DOG-1, E-Cadherin, EGFR, EMA, ER, ERCC1, Factor VIII-RA, Factor XIIIa, Fascin, FoxP1, FoxP3, Galectin-3, GATA-3, GCDFP-15, GCET1, GFAP, Glycophorin A, Glypican 3, Granzyme B, HBME-1, Helicobacter Pylori, Hemoglobin A, Hep Par 1, HER2, HHV-8, HMB-45, HSV 1/11, ICOS, IFNgamma, IgA, IgD, IgG, IgM, IL17, IL4, Inhibin, iNOS, Kappa Ig Light Chain, Ki67, LAG-3, Lambda Ig Light Chain, Lysozyme, Mammaglobin A, MART-1/Melan A, Mast Cell Tryptase, MLH1, MOC-31, MPO, MSA, MSH2, MSH6, MUC1, MUC2, MUM1, MyoD1, Myogenin, Myoglobin, Napsin A, Nestin, NSE, Oct-2, OX40, OX40L, p16, p21, p27, p40, p53, p63, p504s, PAX-5, PAX-8, PD-1, PD-L1, PHH3, PIN-4, PLAP, PMS2, Pneumocystis jiroveci (carinii), PR, PSA, PSAP, RCC, S-100, SMA, SMM, Smoothelin, SOX10, SOX11, Surfactant Apoprotein A, Synaptophysin, TAG 72, TdT, Thrombomodulin, Thyroglobulin, TIA-1, TIM3, TRAcP, TTF-1, Tyrosinase, Uroplakin, VEGFR-2, Villin, Vimentin, and WT-1.

20. The hybridization reagent composition of claim 1, wherein the detectable antibody comprises a detectable label.

21. The hybridization reagent composition of claim 20, wherein the detectable label is a fluorophore, an enzyme, an upconverting nanoparticle, a quantum dot, or a detectable hapten.

22. The hybridization reagent composition of claim 21, wherein the detectable label is a fluorophore.

23. The hybridization reagent composition of claim 21, wherein the enzyme is a peroxidase, an alkaline phosphatase, or a glucose oxidase.

24. The hybridization reagent composition of claim 23, wherein the peroxidase is a horseradish peroxidase or a soybean peroxidase.

25. The hybridization reagent composition of claim 1, wherein the bridging antigen comprises a detectable label.

26. The hybridization reagent composition of claim 25, wherein the detectable label of the bridging antigen is a fluorophore.

27. The hybridization reagent composition of claim 25, wherein the detectable antibody comprises a detectable label.

28. The hybridization reagent composition of claim 27, wherein the detectable label of the bridging antigen and the detectable label of the secondary antibody are both detectable by fluorescence at the same wavelength.

29. The hybridization reagent composition of claim 1, wherein the detectable antibody is specific for the bridging antigen with a dissociation constant of at most 100 nM, at most 30 nM, at most 10 nM, at most 3 nM, at most 1 nM, at most 0.3 nM, at most 0.1 nM, at most 0.03 nM, at most 0.01 nM, or at most 0.003 nM.

30. A multiplexed hybridization reagent composition comprising a plurality of the hybridization reagent compositions of any one of claims 1-29.

31. The multiplexed hybridization reagent composition of claim 30, wherein the composition comprises at least three hybridization reagent compositions.

32. The multiplexed hybridization reagent composition of claim 30, wherein the composition comprises at least five hybridization reagent compositions.

33. The multiplexed hybridization reagent composition of claim 30, wherein the composition comprises at least ten hybridization reagent compositions.

34. A hybridization reagent comprising:
an oligonucleotide probe coupled to a bridging antigen.

35. The hybridization reagent of claim 34, wherein the bridging antigen is a peptide.

36. The hybridization reagent of claim 34, wherein the bridging antigen comprises a plurality of antigenic determinants.

37. The hybridization reagent of claim 36, wherein each antigenic determinant in the plurality of antigenic determinants is the same.

38. The hybridization reagent of claim 36, wherein the plurality of antigenic determinants comprises a linear repeating structure.

39. The hybridization reagent of claim 38, wherein the linear repeating structure is a linear repeating peptide structure.

40. The hybridization reagent of claim 36, wherein the plurality of antigenic determinants comprises at least three antigenic determinants.

41. The hybridization reagent of claim 36, wherein the bridging antigen comprises a branched structure.

42. The hybridization reagent of claim 34, wherein the bridging antigen is a peptide comprising a non-natural residue.

43. The hybridization reagent of claim 42, wherein the non-natural residue is a non-natural stereoisomer.

44. The hybridization reagent of claim 42, wherein the non-natural residue is a β-amino acid.

45. The hybridization reagent of claim 34, wherein the oligonucleotide probe and the bridging antigen are coupled by a chemical coupling reaction through a conjugation moiety.

46. The hybridization reagent of claim 45, wherein the oligonucleotide probe and the bridging antigen are coupled through a high-efficiency conjugation moiety.

47. The hybridization reagent of claim 46, wherein the high-efficiency conjugation moiety is a Schiff base.

48. The hybridization reagent of claim 47, wherein the Schiff base is a hydrazone or an oxime.

49. The hybridization reagent of claim 46, wherein the high-efficiency conjugation moiety is formed by a click reaction.

50. The hybridization reagent of claim 45, wherein the conjugation moiety comprises a cleavable linker.

51. The hybridization reagent of claim 34, wherein the oligonucleotide probe is complementary to at least a segment of a gene encoding a cellular marker or an RNA expressed by the gene.

52. The hybridization reagent of claim 51, wherein the cellular marker is selected from the group consisting of: 4-1BB, AFP, ALK1, Amyloid A, Amyloid P, Androgen Receptor, Annexin A1, ASMA, BCA225, BCL-1, BCL-2, BCL-6, BerEP4, Beta-Catenin, Beta-HCG, BG-8, BOB-1, CA19-9, CA125, Calcitonin, Caldesmon, Calponin-1, Calretinin, CAM 5.2, CD1a, CD2, CD3, CD4, CD5, CD7, CD8, CD10, CD15, CD19, CD20, CD21, CD22, CD23, CD25, CD30, CD31, CD33, CD34, CD38, CD42b, CD43, CD45 LCA, CD45RO, CD56, CD57, CD61, CD68, CD79a, CD99, CD117, CD138, CD163, CDX2, CEA, Chromogranin A, CMV, c-kit, c-MET, c-MYC, Collagen Type IV, Complement 3c (C3c), COX-2, CXCR5, CK1, CK5, CK6, CK7, CK8, CK14, CK18, CK17, CK19, CK20, CK903, CK AE1, CK AE1/AE3, D2-40, Desmin, DOG-1, E-Cadherin, EGFR, EMA, ER, ERCC1, Factor VIII-RA, Factor XIIIa, Fascin, FoxP1, FoxP3, Galectin-3, GATA-3, GCDFP-15, GCET1, GFAP, Glycophorin A, Glypican 3, Granzyme B, HBME-1, Helicobacter Pylori, Hemoglobin A, Hep Par 1, HER2, HHV-8, HMB-45, HSV 1/11, ICOS, IFNgamma, IgA, IgD, IgG, IgM, IL17, IL4, Inhibin, iNOS, Kappa Ig Light Chain, Ki67, LAG-3, Lambda Ig Light Chain, Lysozyme, Mammaglobin A, MART-1/Melan A, Mast Cell Tryptase, MLH1, MOC-31, MPO, MSA, MSH2, MSH6, MUC1, MUC2, MUM1, MyoD1, Myogenin, Myoglobin, Napsin A, Nestin, NSE, Oct-2, OX40, OX40L, p16, p21, p27, p40, p53, p63, p504s, PAX-5, PAX-8, PD-1, PD-L1, PHH3, PIN-4, PLAP, PMS2, Pneumocystis jiroveci (carinii), PR, PSA, PSAP, RCC, S-100, SMA, SMM, Smoothelin, SOX10, SOX11, Surfactant Apoprotein A, Synaptophysin, TAG 72, TdT, Thrombomodulin, Thyroglobulin, TIA-1, TIM3, TRAcP, TTF-1, Tyrosinase, Uroplakin, VEGFR-2, Villin, Vimentin, and WT-1.

53. The hybridization reagent of claim 34, wherein the bridging antigen comprises a detectable label.

54. The hybridization reagent of claim 53, wherein the detectable label is a fluorophore.

55. A multiplexed hybridization reagent composition comprising a plurality of the hybridization reagents of any one of claims 34-54.

56. The multiplexed hybridization reagent composition of claim 55, comprising at least three hybridization reagents.

57. The multiplexed hybridization reagent composition of claim 55, comprising at least five hybridization reagents.

58. The multiplexed hybridization reagent composition of claim 55, comprising at least ten hybridization reagents.

59. A method for hybridization assay comprising:
providing a first sample comprising a first target nucleic acid;
reacting the first target nucleic acid with a first hybridization reagent, wherein the first hybridization reagent is a hybridization reagent of any one of claims 34-54 complementary to the first target nucleic acid;
reacting the first hybridization reagent with a first detectable antibody, wherein the first detectable antibody is specific for the bridging antigen of the first hybridization reagent with high affinity; and
detecting the first detectable antibody that is associated with the bridging antigen of the first hybridization reagent.

60. The method of claim 59, wherein the oligonucleotide probe of the first hybridization reagent is complementary to at least a segment of a gene encoding a cellular marker or an RNA expressed by the gene.

61. The method of claim 60, wherein the cellular marker is selected from the group consisting of: 4-1BB, AFP, ALK1, Amyloid A, Amyloid P, Androgen Receptor, Annexin A1, ASMA, BCA225, BCL-1, BCL-2, BCL-6, BerEP4, Beta-Catenin, Beta-HCG, BG-8, BOB-1, CA19-9, CA125, Calcitonin, Caldesmon, Calponin-1, Calretinin, CAM 5.2, CD1a, CD2, CD3, CD4, CD5, CD7, CD8, CD10, CD15, CD19, CD20, CD21, CD22, CD23, CD25, CD30, CD31, CD33, CD34, CD38, CD42b, CD43, CD45 LCA, CD45RO, CD56, CD57, CD61, CD68, CD79a, CD99, CD117, CD138, CD163, CDX2, CEA, Chromogranin A, CMV, c-kit, c-MET, c-MYC, Collagen Type IV, Complement 3c (C3c), COX-2, CXCR5, CK1, CK5, CK6, CK7, CK8, CK14, CK18, CK17, CK19, CK20, CK903, CK AE1, CK AE1/AE3, D2-40, Desmin, DOG-1, E-Cadherin, EGFR, EMA, ER, ERCC1, Factor VIII-RA, Factor XIIIa, Fascin, FoxP1, FoxP3, Galectin-3, GATA-3, GCDFP-15, GCET1, GFAP, Glycophorin A, Glypican 3, Granzyme B, HBME-1, Helicobacter Pylori, Hemoglobin A, Hep Par 1, HER2, HHV-8, HMB-45, HSV 1/11, ICOS, IFNgamma, IgA, IgD, IgG, IgM, IL17, IL4, Inhibin, iNOS, Kappa Ig Light Chain, Ki67, LAG-3, Lambda Ig Light Chain, Lysozyme, Mammaglobin A, MART-1/Melan A, Mast Cell Tryptase, MLH1, MOC-31, MPO, MSA, MSH2, MSH6, MUC1, MUC2, MUM1, MyoD1, Myogenin, Myoglobin, Napsin A, Nestin, NSE, Oct-2, OX40, OX40L, p16, p21, p27, p40, p53, p63, p504s, PAX-5, PAX-8, PD-1, PD-L1, PHH3, PIN-4, PLAP, PMS2, Pneumocystis jiroveci (carinii), PR, PSA, PSAP, RCC, S-100, SMA, SMM, Smoothelin, SOX10, SOX11, Surfactant Apoprotein A, Synaptophysin, TAG 72, TdT, Thrombomodulin, Thyroglobulin, TIA-1, TIM3, TRAcP, TTF-1, Tyrosinase, Uroplakin, VEGFR-2, Villin, Vimentin, and WT-1.

62. The method of claim 59, wherein the first detectable antibody comprises a detectable label.

63. The method of claim 62, wherein the detectable label is a fluorophore, an enzyme, an upconverting nanoparticle, a quantum dot, or a detectable hapten.

64. The method of claim 63, wherein the detectable label is a fluorophore.

65. The method of claim 63, wherein the enzyme is a peroxidase, an alkaline phosphatase, or a glucose oxidase.

66. The method of claim 65, wherein the peroxidase is a horseradish peroxidase or a soybean peroxidase.

67. The method of claim 59, wherein the first detectable antibody is specific for the bridging antigen of the first hybridization reagent with a dissociation constant of at most 100 nM, at most 30 nM, at most 10 nM, at most 3 nM, at most 1 nM, at most 0.3 nM, at most 0.1 nM, at most 0.03 nM, at most 0.01 nM, or at most 0.003 nM.

68. The method of claim 59, wherein the first target nucleic acid is within a tissue section.

69. The method of claim 68, wherein the detecting step is a fluorescence detection step.

70. The method of claim 68, wherein the detecting step is an enzymatic detection step.

71. The method of claim 59, wherein the first target nucleic acid is in or on a cell.

72. The method of claim 71, wherein the first target nucleic acid is in the cytoplasm of the cell.

73. The method of claim 71, wherein the first target nucleic acid is in the nucleus of the cell.

74. The method of claim 71, wherein the detecting step is a fluorescence detection step.

75. The method of claim 74, further comprising:
sorting cells that have bound the first detectable antibody.

76. The method of claim 59, further comprising:
reacting a second target nucleic acid on the first sample with a second hybridization reagent, wherein the second hybridization reagent is a hybridization reagent of any one of claims 34-54 complementary to the second target nucleic acid;
reacting the second hybridization reagent with a second detectable antibody, wherein the second detectable antibody is specific for the bridging antigen of the second hybridization reagent with high affinity; and
detecting the second detectable antibody that is associated with the bridging antigen of the second hybridization reagent.

77. The method of claim 76, further comprising:
detecting at least three target nucleic acids in the sample.

78. The method of claim 76, further comprising:
detecting at least five target nucleic acids in the sample.

79. The method of claim 76, further comprising:
detecting at least ten target nucleic acids in the sample.

80. The method of claim 59, further comprising:
reacting a second target nucleic acid on a second sample with a second hybridization reagent, wherein the second hybridization reagent is a hybridization reagent of any one of claims 35-56 complementary to the second target nucleic acid;
reacting the second hybridization reagent with a second detectable antibody, wherein the second detectable antibody is specific for the bridging antigen of the second hybridization reagent with high affinity; and
detecting the second detectable antibody that is associated with the bridging antigen of the second hybridization reagent; wherein the first sample and the second sample are serial sections of a tissue sample.

81. The method of claim 80, wherein a plurality of target nucleic acids are detected on the first sample and a plurality of target nucleic acids are detected on the second sample.

82. The method of claim 81, wherein at least three target nucleic acids are detected on the first sample and at least three target nucleic acids are detected on the second sample.

83. The method of claim 80, wherein at least three target nucleic acids are detected on at least three samples, and wherein the at least three samples are serial sections of a tissue sample.

84. The method of claim 83, wherein a plurality of target nucleic acids are detected on each of the at least three samples.

85. The method of claim 84, wherein at least three target nucleic acids are detected on each of the at least three samples.

86. A method for hybridization assay comprising:
providing a sample comprising a first target nucleic acid;
reacting the first target nucleic acid with a first hybridization reagent, wherein the first hybridization reagent is a hybridization reagent of any one of claims 34-54 complementary to the first target nucleic acid;
reacting the first hybridization reagent with a first reactive antibody, wherein the first reactive antibody binds to the bridging antigen of the first hybridization reagent with high affinity; and
reacting the first reactive antibody with a first detectable reagent, wherein the first detectable reagent is bound to the sample in proximity to the first target nucleic acid.

87. The method of claim 86, wherein the first reactive antibody comprises an enzyme activity.

88. The method of claim 87, wherein the enzyme activity is a peroxidase activity.

89. The method of claim 88, wherein the peroxidase activity is a horse radish peroxidase activity.

90. The method of claim 86, wherein the first detectable reagent comprises a tyramide.

91. The method of claim 86, wherein the first detectable reagent comprises a fluorophore or a chromophore.

92. The method of claim 86, further comprising:
dissociating the first reactive antibody from the sample.

93. The method of claim 92, wherein the first reactive antibody is dissociated from the sample by a selective treatment.

94. The method of claim 93, wherein the selective treatment comprises treatment with a soluble bridging antigen.

95. The method of claim 93, wherein the selective treatment comprises cleavage of a cleavable linker.

96. The method of claim 92, wherein the first reactive antibody is dissociated from the sample by a heat treatment.

97. The method of claim 92, further comprising:
reacting a second target nucleic acid on the sample with a second hybridization reagent, wherein the second hybridization reagent is a hybridization reagent of any one of claims 34-54 complementary to the second target nucleic acid;
reacting the second hybridization reagent with a second reactive antibody, wherein the second reactive antibody binds to the bridging antigen of the second hybridization reagent with high affinity; and
reacting the second reactive antibody with a second detectable reagent, wherein the second detectable reagent is bound to the sample in proximity to the second target nucleic acid.

98. The method of claim 97, wherein the second reactive antibody comprises an enzyme activity.

99. The method of claim 98, wherein the enzyme activity is a peroxidase activity.

100. The method of claim 99, wherein the peroxidase activity is a horse radish peroxidase activity.

101. The method of claim 97, wherein the second detectable reagent comprises a tyramide.

102. The method of claim 97, wherein the second detectable reagent comprises a fluorophore or a chromophore.

103. The method of claim 97, wherein the first reactive antibody is dissociated from the sample by a selective treatment.

104. The method of claim 103, wherein the selective treatment comprises treatment with a soluble bridging antigen.

105. The method of claim 103, wherein the selective treatment comprises cleavage of a cleavable linker.

106. The method of claim 97, wherein the first reactive antibody is dissociated from the sample by heat treatment.

107. The method of claim 97, further comprising:
detecting the first detectable reagent and the second detectable reagent on the sample.

108. A kit for hybridization assay comprising:
the hybridization reagent of any one of claims 34-54;
a detectable antibody specific for the bridging antigen with high affinity; and
instructions for using the kit.

109. The kit of claim 108, wherein the detectable antibody comprises a detectable label.

110. The kit of claim 109, wherein the detectable label is a fluorophore, an enzyme, an upconverting nanoparticle, a quantum dot, or a detectable hapten.

111. The kit of claim 110, wherein the detectable label is a fluorophore.

112. The kit of claim 111, wherein the enzyme is a peroxidase, an alkaline phosphatase, or a glucose oxidase.

113. The kit of claim 112, wherein the peroxidase is a horseradish peroxidase or a soybean peroxidase.

114. The kit of claim 108, wherein the detectable antibody is specific for the bridging antigen with a dissociation constant of at most 100 nM, at most 30 nM, at most 10 nM, at most 3 nM, at most 1 nM, at most 0.3 nM, at most 0.1 nM, at most 0.03 nM, at most 0.01 nM, or at most 0.003 nM.

115. The kit of claim 108, comprising:
at least three hybridization reagents of any one of claims 34-54;
at least three detectable antibodies specific for the bridging antigens with high affinity; and
instructions for using the kit.

116. The kit of claim 108, comprising:
at least five hybridization reagents of any one of claims 34-54;
at least five detectable antibodies specific for the bridging antigens with high affinity; and
instructions for using the kit.

117. The kit of claim 108, comprising:
at least ten hybridization reagents of any one of claims 34-54;
at least ten detectable antibodies specific for the bridging antigens with high affinity; and
instructions for using the kit.
